(19) United States
(12) Patent Application Publication
(10) Pub. No.: US 2004/0067578 A1
(43) Pub. Date: Apr. 8, 2004

(54) Electrospray interface

(76) Inventor: Jan Axelsson, Storvreta (SE)

Correspondence Address: AMERSHAM BIOSCIENCES, PATENT DEPARTMENT, 800 CENTENNIAL AVENUE, PISCATAWAY, NJ, 08855, US

(21) Appl. No.: 10/432,514

(22) PCT Filed: Dec. 4, 2001
(86) PCT No.: PCT/EP01/14190

(30) Foreign Application Priority Data
Dec. 8, 2000 (SE) 0004574-0

(51) Int. Cl. C12M 1/34; H01J 49/00; B01D 59/44

(52) U.S. Cl. 435/287.2; 250/281

(57) ABSTRACT

The present invention relates to an electrospray interface (13) for a microchannel device having a body (1) comprising at least one microchannel (7) with an opening (9A-9C) wherein the opening is provided with a plurality of fluid dispersing means (15A, 15B).

### FIELD OF THE INVENTION

The present invention relates to devices of the type mentioned in the preamble of the independent claim for use in electrospraying.

### PRIOR ART

Mass spectrometers are often used to analyse the masses of components of liquid samples obtained from analysis devices such as liquid chromatographs. Mass spectrometers require that the component sample that is to be analysed be provided in the form of free ions and it is usually necessary to evaporate the liquid samples in order to produce a vapour of ions. This is commonly achieved by using electrospray ionisation. In electrospray ionisation (ESI) applying a voltage (in the order of 2-6 kV) to a hollow needle through which the liquid sample can freely flow generates a spray. The inlet orifice to the mass spectrometer is given a lower potential, for example 0V, and an electrical field is generated from the tip of the needle to the orifice of the mass spectrometer. The electrical field attracts the positively charge species in the fluid which accumulate in the meniscus of the liquid at the tip of the needle. The negatively charged species in the fluid are neutralised. This meniscus extends towards the oppositely charged orifice and forms a “Taylor cone”. When the attraction between the charged species and the orifice exceeds the surface tension of the tip of the Taylor cone, droplets break free from the Taylor cone and fly in the direction of the electrical field lines into the orifice of the mass spectrometer where analysis of the species takes place.

Microfluid chip devices have been developed to enable high throughput analysis of very small volumes of samples. These devices have one or more channels with a width of only a few micrometers and attempts have been made to use the outlets of such channels as electrospray interface tips. An example of this can be found in U.S. Pat. No. 5,969,353, which describes an interface tip attached to, or produced on, an outlet port of a microfluid chip. These tips, however, are difficult to attach, respectively produce, and are fragile.

### SUMMARY OF THE INVENTION

According to the present invention, at least some of the problems with the prior art are solved by means of a device having the features present in the characterising part of claim 1. Further advantages and improvements can be obtained by means of devices having the features mentioned in the dependent claims.

### BRIEF DESCRIPTION OF THE FIGURES

FIG. 1 shows a perspective view of a microchannel device provided with interfaces in accordance with the present invention;

FIG. 2 shows an enlarged view of a first type of interface in accordance with the present invention;

FIG. 3 shows an enlarged view of a second type of interface in accordance with the present invention; and

FIG. 4 shows an enlarged view of a third type of interface in accordance with the present invention.

### DETAILED DESCRIPTION OF EMBODIMENTS ILLUSTRATING THE INVENTION

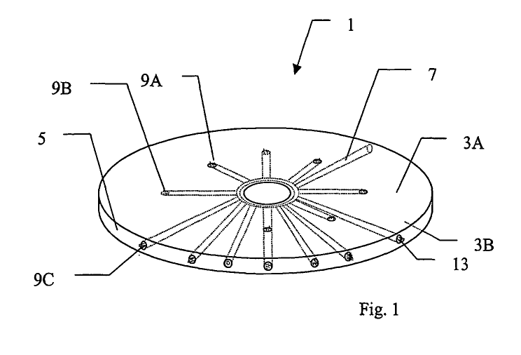

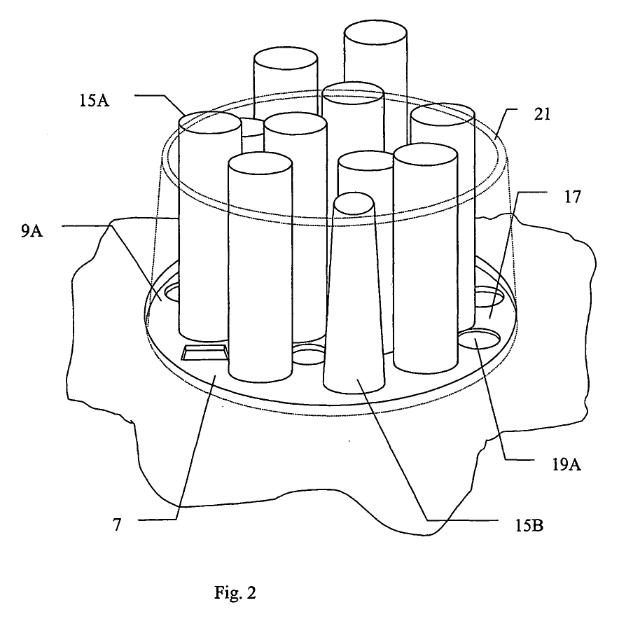

FIG. 1 shows a perspective view, not to scale, of the body 1 of a microchannel device having a top surface 3A, a bottom surface 3B and a peripheral wall 5. Device 1 has a plurality of microchannels 7, which lead from the centre of the device 1 to openings 9A in the top surface 3, openings 9B in the bottom surface 3A and openings 9C in the wall 5 of the device 1. The openings 9A-9C are intended to allow fluid inside the microchannels to be extracted from the microchannels. The width of an opening, or its diameter in the case of round openings, depends on the intended flow rate through it, which can be from about 1 μl per hour upwards, and can vary from about 0.1 μm upwards. Openings 9A-9C are provided with interfaces 13 in accordance with the present invention. As can be seen from FIG. 2, an interface 13 in accordance with a first embodiment of the present invention is formed of a plurality of fluid dispersing means in the form of strands 15A, 15B, which project from an opening 9A. Strands 15A, 15B are solid and form a brush-like structure. Strands 11A are substantially cylindrical, while strand 15B is tapered. Typically a strand 15A, 15B is between about 0.1 μm and 50 μm wide and projects from about 0.1 μm to 2 mm from the opening. If the opening is 2 mm wide then the longest strand 15A, 15B can project about 2 mm from the opening. If the opening is 0.1 mm wide then a suitable length for the longest strand could be 0.1 mm. When selecting the length of strands, it can be important to consider the volume of the spaces between, or within, the strands. If the volume is made small then the width of the detected peaks will be reduced which is desirable. However, if the volume between the strands is too small then the resistance to fluid flow will be high and analysis times will be increased. Therefore a compromise may have to be made between peak width and fluid flow. The lengths of the strands used can be varied in order to keep the volume of fluid between the strands small while at the same time achieving a stable Taylor cone and a stable spray jet of droplets. Strands 15A and 15B can be of different length, in which case it can be advantageous to arrange the taller strands in the middle of the opening 9A with progressively smaller strands towards the edge of opening 9A so that the tips of the strand form points on the surface of an imaginary cone or pyramid. If the tallest strand is 10 μm high and the diameter of the opening is 10 μm then the volume of a regular cone with a height of 10 μm would be around 0.5 pl. Strands may be bonded or formed together to form a bunch of strands which is bonded or otherwise attached to the perimeter of opening 9A. Alternatively, opening 9A is preferably provided with a dispersing means-supporting surface 17 that supports strands 15A, 15B. In order to allow fluid to exit the microchannel 7, strand supporting surface 17 is provided with one or more fluid outlet orifices 19A sufficiently large to allow fluid inside the microchannel 7 to exit the microchannel. This fluid forms a meniscus that covers the strands 15A, 15B. When used in an electrospray device, the fluid forms a Taylor cone under the influence of the electrospray electrical field. Optionally, the lengths of the strands 15A, 15B can be adapted so that the tips of the strands 15A, 15B, form a conical shape which preferably mirrors the surface of the Taylor cone. In order to protect the fluid dispersing means from damage, they can be surrounded by a protective wall 21 (shown by a dotted line). This wall can be constructed from the same material as the body 1 or strands 15A, 15B, or be formed from, for example, a liquid varnish that can be painted around the strands and allowed to dry. The viscosity of the liquid varnish and its surface tension should be chosen so that the varnish does not flow between the strands, in order to leave the spaces between the strands 15A, 15B free for the fluid coming out of the orifices 19A.

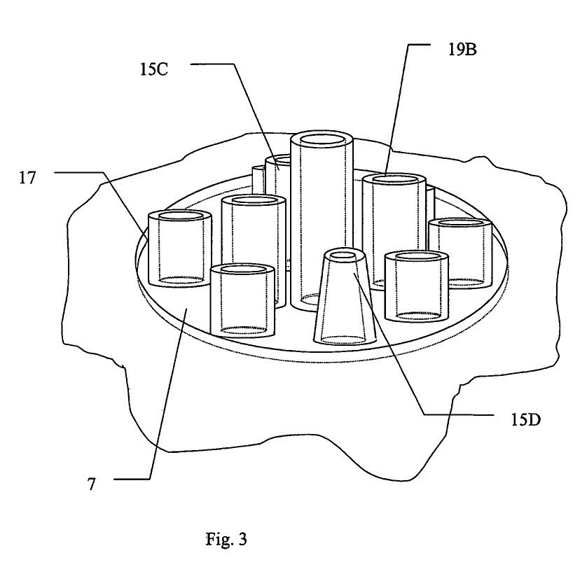

FIG. 3 shows a second embodiment of the present invention. In this embodiment the fluid dispersing strands 15C, 15D are hollow and have a fluid outlet orifice 19B at the end furthest away from body 1. Fluid can exit microchannel 7 by flowing out through the strands 15C, 15D.

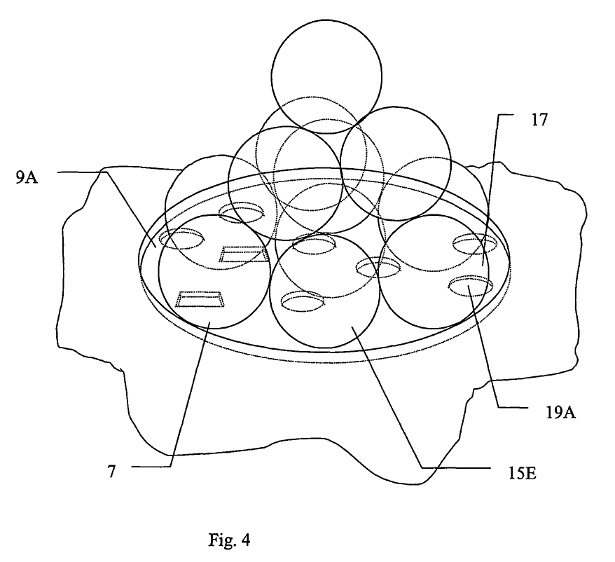

FIG. 4 shows a third embodiment of the present invention. In this embodiment the fluid dispersing means is in the form of beads 15E which are piled on top of each other. In the example shown in FIG. 4, the beads 15E are piled up to form a cone, with the lowest layer of beads 15E being joined to the supporting surface 17. Fluid can exit microchannel 7 by flowing out through the outlets 19 and can then travel further on the outer surfaces of the beads. The beads 15E can be of differing sizes and do not have to be spherical but can be ovoid or even irregularly shaped.

Microchannel device 1 can be made of any suitable material such as silicon, glass, plastic, etc. Dispersing means 15A-15E can be made of any suitable material such as silicon, glass, plastic, metal etc. Dispersing means 15-15E can be made in situ by any suitable sort of micromachining or micromanufacturing process which would leave the desired structure e.g. casting, etching, laser machining, deposition of material by plating, precipitation or spraying/printing, micromilling, reducing the diameter of tubes or cylinders by heating and stretching, etc.

Dispersing means 15A-15E may also be made separately and attached to the body 1 one at a time or after having been assembled into a bunch of strands or cone of beads. Dispersing means 15A-15E can be attached to each other and to the body 1 by any suitable means such as adhesion, welding, interference fitting, etc.

The diameters of the distal ends of strands 15A-15D can be adapted to the flow rates required with smaller ends allowing an even flow at low flow rates. Larger distal ends give an even flow at higher flow rates that would saturate the smaller ends and cause the fluid to coalesce into irregularly sized drops. Strands could have lengths of 0.1 μm upwards, outside diameters from 1 μm upwards and, where applicable, inside diameters from 0.5 μm upwards. Beads 15E can have diameters from 0.1 μm upwards. Preferably the length of strands and the diameters of beads is less than 1 mm in order to keep the interface as compact as possible and to minimise dead volumes.

Dispersing means can be provided with coatings or can be constructed so that they act on the fluid passing through or by them. The coating or construction can be adapted to improve the quality of the fluid by removing unwanted fractions or particles in the fluid. For example, strands and beads can be coated with an agent for, e.g. absorbing salts or proteins from the fluid, or can be made porous to act as filters for trapping particles in the fluid which have a size greater than the size of the pores.

In accordance with the present invention, it is also conceivable to provide a microchannel device with interfaces that comprise at least one hollow fluid dispensing strand and at least one solid fluid dispensing strand and/or at least one fluid dispensing bead.

It is furthermore conceivable to provide a microchannel device with nebulising means, such as a source of ultrasonic waves, which can cause the dispensing means to shake or vibrate and hereby promote nebulisation of the fluid.

The above mentioned embodiments are intended to illustrate the present invention and are not intended to limit the scope of protection claimed by the following claims.

1. An electrospray interface (13) for a microchannel device having a body (1) comprising at least one microchannel (7) with an opening (9A-9C), characterised in that said opening is provided with a plurality of fluid dispersing means (15A-15E), wherein at least one of said fluid dispersing means (15A-15E) is a projection (15A, 15B).

2. An electrospray interface in accordance with claim 1 characterised in that at least one of said fluid dispensing means (15A-15E) is solid (15A, 15B).

3. An electrospray interface in accordance with claim 1 characterised in that at least one of said fluid dispersing means is hollow (15C, 15D).

4. An electrospray interface in accordance with any of the previous claims characterised in that at least one of said fluid dispersing means (15A-15E) is a solid bead (15E).

5. An electrospray interface in accordance with any of the previous claims characterised in that the minimum width of a fluid dispersing strand (15A-15D) or the minimum diameter of a fluid dispersing bead (15E) is 0.1 μm, and the maximum width or diameter of a strand or bead is 1 mm.

6. An electrospray interface in accordance with any of the previous claims characterised in that the minimum length of a fluid dispersing strand (15A-15D) is 0.1 μm and the maximum length of a fluid dispersing strand is 1 mm.

7. An electrospray interface in accordance with any of the previous claims characterised in that the fluid dispersing means (15A-15E) is made of the same material as the body (1).

8. An electrospray interface in accordance with any of claims 1-6 characterised in that the fluid dispersing means (15A-15E) is made of a different material to the material that the body (1) is made from.

9. An electrospray interface in accordance with any of the previous claims characterised in that said fluid dispensing means (15A-15E) is provided with a coating or construction suitable for absorbing chemicals or trapping particles.

10. An electrospray interface in accordance with any of the previous claims characterised in that it is provided with a source of ultrasonic waves.
